(19) United States
(12) Patent Application Publication
(10) Pub. No.: US 2022/0331613 A1
(43) Pub. Date: Oct. 20, 2022

(54) WEARABLE ULTRASONIC TREATMENT DEVICE

(71) Applicant: One Chica Medical Devices, LLC, Austin, TX (US)

(72) Inventor: Bonnie Teal Sullivan, Austin, TX (US)

(21) Appl. No.: 17/713,925

(22) Filed: Apr. 5, 2022

Related U.S. Application Data
(60) Provisional application No. 63/175,892, filed on Apr. 16, 2021.

(51) Int. Cl. A61N 7/00

(52) U.S. Cl. CPC A61N 7/00; A61N 2007/0017; A61N 2007/0078

(57) ABSTRACT

A portable, light-weight ultrasound therapy system includes a wearable garment and a flexible ultrasound device. The flexible ultrasound device includes a plurality of piezoelectric transducers. A control system is configured to couple the flexible ultrasound device to an external processing system configured to control operation of the plurality of piezoelectric transducers.

### CROSS-REFERENCE TO RELATED APPLICATION AND CLAIM OF PRIORITY

This application claims priority under 35 U.S.C. § 119(e) to U.S. Provisional Patent Application No. 63/175,892 filed on Apr. 16, 2021. The above-identified provisional patent application is hereby incorporated by reference in their entireties

TECHNICAL FIELD

The present disclosure relates to a medical device for the treatment of scar tissue. More specifically, this disclosure relates to a wearable ultrasonic device for the treatment of capsular contracture and other scar tissue resulting from surgery.

### BACKGROUND

Capsular contracture, also referenced as capsular contraction, is a response of the immune system to foreign materials in the human body. A capsule of “scar tissue” may form around a medical or cosmetic implant. The formation of the scar tissue is a natural immune reaction of the body to isolate the implant within the body by creating a barrier of scar tissue around the implant. Most commonly, capsular contracture results from complications with regards to breast augmentation surgery as well as surgeries for artificial joint prosthetics. Additionally, arthrofibrosis is the buildup of scar tissue around a joint, usually resulting after a traumatic injury or surgical procedure. Arthrofibrosis, also referenced as stiff knee syndrome, may limit a range of motion and cause pain and disability.

As much as 15-40% of patients will experience some form of capsular contracture after surgery. Generally, capsular contracture occurs during the healing process. Approximately 75% of capsular contractures will occur within two years of the implants being placed. The resulting scarring makes the tissue firm or hard. The capsular contracture can be measured on a Baker scale of I to IV. The Baker grades for breast augmentation are:

Grade I—the breast is normally soft and appears natural in size and shape—grade one capsular contracture is asymptomatic (producing or showing no symptoms). The formation of scar tissue around the implant does not interfere with the size, shape or texture of the breasts.

Grade II—the breast is a little firm; but appears normal—Grade two capsular contracture usually presents itself with only minor cosmetic symptoms. The breasts will usually appear normal in shape but feel somewhat firm to the touch.

Grade III—the breast is firm and appears abnormal; Grade three capsular contracture presents itself with obvious cosmetic symptoms. The breasts will be firm to the touch and appear abnormal. For example, they may appear overly round and hard-looking. However, this grade of capsular contraction often doesn't cause much (if any) pain.

Grade IV—the breast is hard, painful to the touch, and appears abnormal—Like grade three capsular contracture, grade four capsular contracture causes the breasts to become hard and misshapen. Patients with grade four capsular contracture also experience breast soreness; their breasts will often be tender and painful to the touch.

Current methods for treating capsular contracture or arthrofibrosis include extensive physical therapy as well as massage therapy for the affected region. Using physical therapy or massage therapy can be cumbersome, time consuming, and inefficient. For example, physical therapy often requires the scheduling of an appointment as well as planning the time and distance to find a qualified and skilled physical therapist. Additionally, self-massage may be ineffective if the patient performs the massage poorly or too lightly due to pain resulting from the massage. Subsequent surgeries may also be required to correct for resulting capsular contracture or arthrofibrosis.

### SUMMARY

The present disclosure illustrates embodiments of portable, wearable ultrasound therapy system.

In a first embodiment, a portable, light-weight ultrasound therapy system is provided. The ultrasound therapy system includes a wearable garment and a flexible ultrasound device. The flexible ultrasound device includes a plurality of piezoelectric transducers. A control system is configured to couple the flexible ultrasound device to an external processing system configured to control operation of the plurality of piezoelectric transducers.

In a second embodiments, a wearable garment is provided. The wearable garment includes a fabric housing configured to be worn on a body portion of a patient. The wearable garment also includes a flexible ultrasound device configured to emit an ultrasonic energy a target site below a skin surface corresponding to the body portion of the patient. The wearable garment further includes a control system configured to operate the plurality of transducers to direct the ultrasonic energy into the target site.

In a third embodiment, a method is provided. The method includes providing a flexible ultrasound device to be positioned on a surface of a body of a patient by a wearable garment, the flexible ultrasound device comprising a plurality of transducers. The method also includes controlling an operation of the plurality of transducers to direct the ultrasonic energy into a target site beneath a skin of the patient.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an ultrasound device according to embodiments of the present disclosure;

FIG. 2 illustrates a wearable ultrasonic therapy sleeve according to embodiments of the present disclosure;

FIG. 3 illustrates a wearable ultrasonic therapy garment according to embodiments of the present disclosure;

FIG. 4 illustrates a controller for use with a wearable ultrasound device according to embodiments of the present disclosure;

FIG. 5 illustrates an external electronic device for controlling a wearable ultrasonic system according to embodiments of the present disclosure; and

FIG. 6 illustrates a process for providing ultrasonic therapy by a wearable ultrasonic device according to embodiments of the present disclosure.

### DETAILED DESCRIPTION

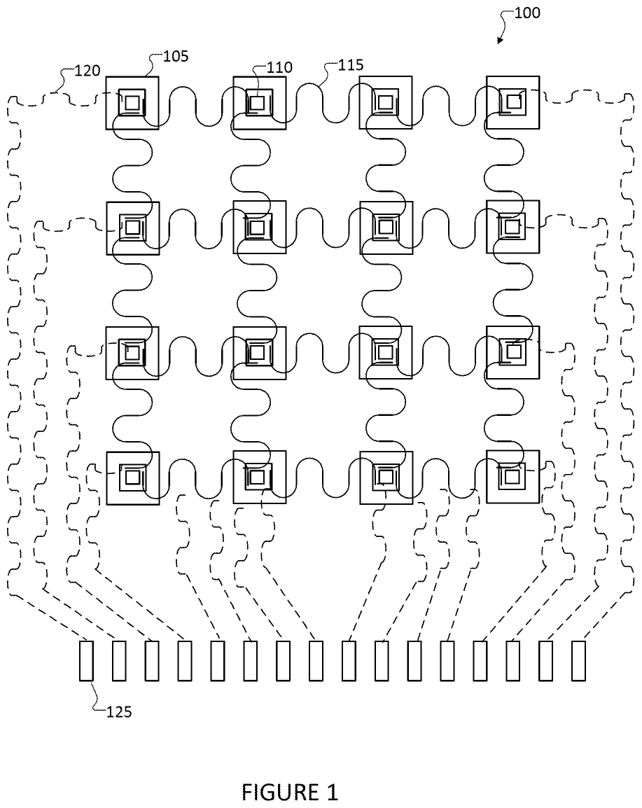

FIGS. 1 through 6, discussed below, and the various embodiments used to describe the principles of the present disclosure are by way of illustration only and should not be construed in any way to limit the scope of the disclosure. Those skilled in the art will understand that the principles of the present disclosure may be implemented in any suitably arranged system or device.

Ultrasonic technology has been widely used for medical technology, including imaging various organs in a body as well as for the diagnosis of potential issues. An ultrasonic device includes a number of transducers operating at a specified frequency and power according to the desired usage and parameters, such as patient parameters (such as age, body size, etc.), and the organ or region of the body to be imaged. Ultrasound also reduces scarring. For example, ultrasound can be used as part of a physical therapy regime before or after scarring develops.

Embodiments of the present disclosure provide safe, comfortable, at-home post-operation (post-op) ultrasound therapy to reduce scar tissue that may limit mobility or cause discomfort. Certain embodiments provide a wearable device or garment that emits ultrasonic waves to inhibit the growth of scar tissue resulting from capsular contracture or arthrofibrosis. Certain embodiments provide an ultrasound device that is attachable onto or insertable into a wearable sleeve or garment.

FIG. 1 illustrates a stretchable and miniature sized, ultrasound device according to embodiments of the present disclosure. The embodiment of the ultrasound device 100 shown in FIG. 1 is for illustration only. In certain embodiments, the ultrasound device 100 can be configured the same as, or similar to, the stretchable ultrasonic transducer array described in U.S. patent application Ser. No. 16/477,060, filed on Jul. 10, 2019, the contents of which are incorporated herein by reference. Other embodiments could be used without departing from the scope of the present disclosure.

The ultrasound device 100 comprises a flexible and stretchable structure and includes a number of transducers 105. In certain embodiments, each transducer 105 can include a substrate, a first patterned bilayer that includes a polymide layer and an electrode, a piezoelectric electric, a backing layer, a second patterned bilayer that includes a polymide layer and an electrode 110, and a superstrate. The elastomer substrate and superstrate thickness are 15 micrometers (μm). Each of the transducers 105 can be configured as a rigid, or semi-rigid electronic element.

Each of the transducers 105 is coupled to one or more neighboring transducers 105 via a first wired line 115. The first wired line 115 can be configured to be bendable or extendable. For example, the first wired line 115 can be configured in a coiled or serpentine shape to expand when needed in a stretched or expanded state and return to a coiled form at a neutral state. Each of the transducers 105 is further coupled via a second wired line 120 to a control interconnects 125. The second wired line can be configured in a coiled or serpentine shape. In certain embodiments, one or more electrodes 110 are stacked in layers to enable multiple connections for the first wired line 115 and second wired line 120. In certain embodiments, one of electrodes is coupled to a common ground layer.

The transducers 105 are disposed at a nominal spacing to enable the ultrasound device 100 to be very small but spaced far enough to enable to first wired line 115 and second wire line 120 to retain enough coiling to provide enhanced stretchability. For example, in certain embodiments, a 10×10 array of transducers 105 is dimensioned to be 1 centimeter (cm)×1 cm. That is, in certain embodiments, a second of the ultrasound device 100 is configured to include 100 transducers 105 within a 1 cm² square area. In certain embodiments, the ultrasound device 100 is dimensioned to be larger than 1 cm², such as 100 cm², but include a ratio of 100 transducers per 1 cm².

The ultrasound device 100 includes a silicone elastomer substrate and a silicone elastomer superstrate. The silicon elastomer provides insultation between and around transducers and wires 115, 120. The silicon elastomer encapsulates the ultrasound device 100 and provides a water-resistant layer configured to protect the electronic elements of the ultrasound device 100. The silicon elastomer also enables the ultrasound device 100 to be flexible and stretchable.

In certain embodiments, an anisotropic conductive film (ACF) is bonded to the control interconnects 125. In certain embodiments, the control interconnects 125 comprise copper (Cu) interconnects. The ACF bonded to the control interconnects 125 offers conductive access to external power supplies and data acquisition.

In certain embodiments, the wired line 115 or wired line 120, or both, comprise a flexible, extendable, or both, connection. The substrate and the configuration flexible or extendable connections enable the ultrasound device 100 to be stretched while still being able to return to its original dimensions and configuration. As such, each of the transducers 105 can be flexibly connected to a neighboring transducer 105. Because the wired lines 115 or 120 is flexible and extendable, the connections further enable the ultrasonic device 100 to be stretchable. That is, distances between neighboring transducers 105 can be extended during an application and returned to a nominal distance when not in the application. In certain embodiments, the flexible connection enables a plurality of flexibly connected transducers to form a conformable ultrasound device 100. The conformable ultrasound device 100 is configured to conform, bend, or match a contour of bones and soft tissue in a body of a patient. For example, the conformable ultrasound device 100 is configured to conform to one or more body portions of a user, such as by matching the contour of the soft tissue and place a plurality of transducers 105 along the contours of the soft tissue. The conformable ultrasound device 100 is configured and dimensioned such that a plurality of transducers 105 can be on each surface of the soft tissue and bend and curve along the surface of the soft tissue. In certain embodiments, the conformable ultrasound device 100 can apply a sonic energy along the surface of the soft tissue. In certain embodiments, the conformable ultrasound device 100 can control the transducers 105 individually or in sections to apply sonic energy to specific surfaces or portions of the soft tissue.

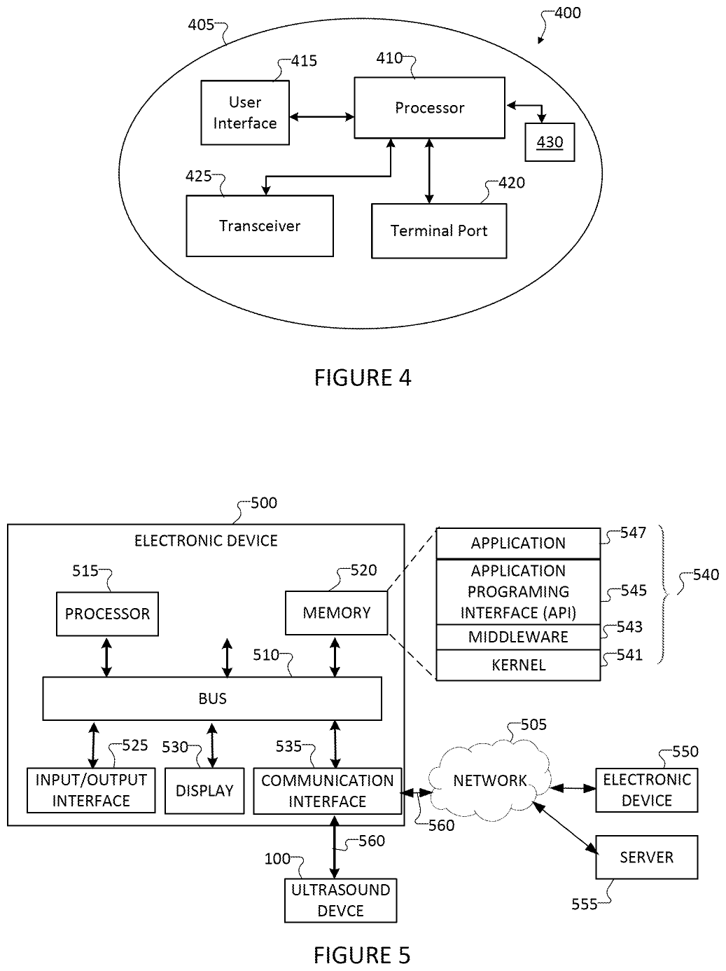

Each of the transducers 105 is configured to emit an ultrasonic energy. In certain embodiments, each the conformable ultrasound device 100 includes a plurality of transducers 105 configured in a grid, such as an N×M grid, where N and M are integers. In certain embodiments, the plurality of transducers 105 is coupled to a control system, such as controller 400 described in further detail with respect to FIG. 4. In certain embodiments, one or more of the transducers 105 can be controlled separately from others of the plurality of transducers 105. For example, a first subset of transducers 105 can be activated to emit the ultrasonic energy while a second subset of the transducers 105 is controlled to remain inactive such that the second subset does not emit the ultrasonic energy while the first subset is emitting the ultrasonic energy. Accordingly, in certain embodiments, each of the transducers includes an individual connection, such as a control line, to the control system. In certain embodiments, respective subsets of the plurality of transducers includes a subset connection, such as a control line to the subset, to the control system.

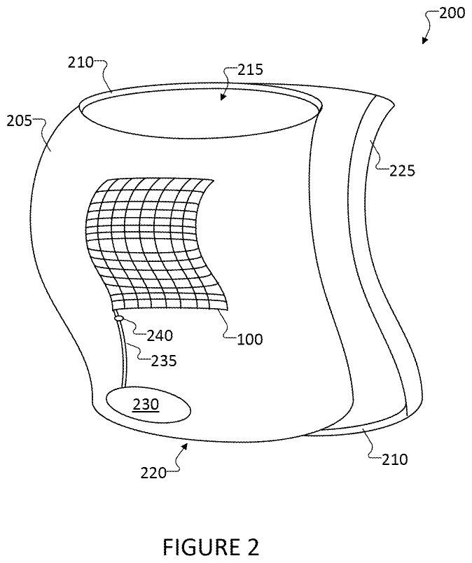

FIG. 2 illustrates a wearable ultrasonic therapy sleeve according to embodiments of the present disclosure. The wearable ultrasonic therapy sleeve 200 shown in FIG. 2 is for illustration only. Other embodiments could be used without departing from the scope of the present disclosure.

In the example depicted in FIG. 2, the wearable ultrasonic therapy sleeve 200 is configured as a knee-brace. It will be understood that the wearable ultrasonic therapy sleeve 200 can be configured in other forms or shapes, such as a sleeve to cover an elbow, a sleeve configured to fit a shoulder, or a wrap around an arm, a leg, or a torso section of the body.

The wearable ultrasonic therapy sleeve 200 includes a fabric housing, such as a stretchable, breathable, fabric material 205. The stretchable fabric material can include a knitted design of one or more of: NEOPRENE, SPANDEX, NYLON, or latex fiber. The wearable ultrasonic therapy sleeve 200 can also include a non-slip layer 210. The non-slip layer can 210 include a non-slip material, such as silicon. In certain embodiments, the non-slip layer 210 includes two or more strips of non-slip material.

The wearable ultrasonic therapy sleeve 200 can be configured as a high compression sleeve or brace that is configured to support a joint. For example, the wearable ultrasonic therapy sleeve 200 can be one of: a knee brace, an elbow brace, an ankle brace, a foot brace, a hand brace, a shoulder brace, a back posture brace, a hip wrap or brace, or a brassiere. In certain embodiments, the wearable ultrasonic therapy sleeve 200 is formed as an enclosed cylindrical sleeve having an opening 215 on a top portion and an opening 220 on a bottom portion. In the enclosed cylindrical sleeve configuration, the wearable ultrasonic therapy sleeve 200 is configured to slide onto a joint or region of the body for treatment, and easily removed by being slid off after treatment. In certain embodiments, the wearable ultrasonic therapy sleeve 200 is configured to open and close for application and removal. In certain embodiments, the ultrasonic therapy sleeve 200 includes an adhesive means 225 to enable the ultrasonic therapy sleeve 200 to be held in the closed position. The adhesive means 225 can include one or more of: a hook and loop structure (such as VELCRO), a zipper, buttons, clips, hooks, fasteners, or a combination thereof.

In certain embodiments, the ultrasonic therapy sleeve 200 includes the ultrasound device 100. In certain embodiments, the ultrasonic therapy sleeve 200 is configured to position the ultrasound device 100 to be non-invasively positioned proximate a skin surface of the patient above a target site below the skin surface. The ultrasound device 100 can be dimensioned to cover a substantial area of the ultrasonic therapy sleeve 200 to ensure full coverage over an affected or treatment area. For example, the therapy sleeve 200 can be dimensioned to cover a 20 cm×20 cm area. It will be understood that the example of a 20 cm×20 cm area is for illustration and explanation only and larger or smaller sizes could be used. Regardless of the size of the ultrasound device 100, the ultrasound device 100 includes 100 transducers per 1 cm².

In certain embodiments, the ultrasound device 100 is permanently attached to the ultrasonic therapy sleeve 200. For example, the ultrasound device 100 can be woven within fibers of the fabric material 205 or sewn onto the fabric material 205. In certain embodiments, the ultrasound device 100 is affixed to the fabric material 205 by an adhesive, such as a silicon adhesive, epoxy, or other glue.

In certain embodiments, the ultrasound device 100 is removably attachable to the ultrasonic therapy sleeve 200. In certain embodiments, the ultrasonic therapy sleeve 200 includes one or more pockets configured to hold the ultrasound device 100. For example, the ultrasonic therapy sleeve 200 can have one pocket to hold one ultrasound device 100 or have two pockets to hold two different ultrasonic devices 100. In certain embodiments, the ultrasound device 100 includes an adhesive material configured to enable the ultrasound device 100 to be attached to different portions of the ultrasonic therapy sleeve 200. In certain embodiments, the ultrasound device 100 includes a coupling means such as snaps, buttons, hooks and loops, or adhesives.

In certain embodiments, the ultrasonic therapy sleeve 200 includes a controller 230. The controller 230 can be coupled to the ultrasound device 100 via a wireline 235, such as an ACF. In certain embodiments, the controller 230 is removably coupled to the ultrasound device 100. For example, the wireline 235 can include a coupling 240, such as an electrical terminal or data terminal. In certain embodiments, the controller 230 is removably coupled to the wireline 235 and ultrasonic therapy sleeve 200. For example, the controller 230 can be removable in the event that the ultrasonic therapy sleeve 200 is to be washed. In certain embodiments, the wireline 235 includes a number of control lines individually coupled to respective transducers or to respective sets of transducers.

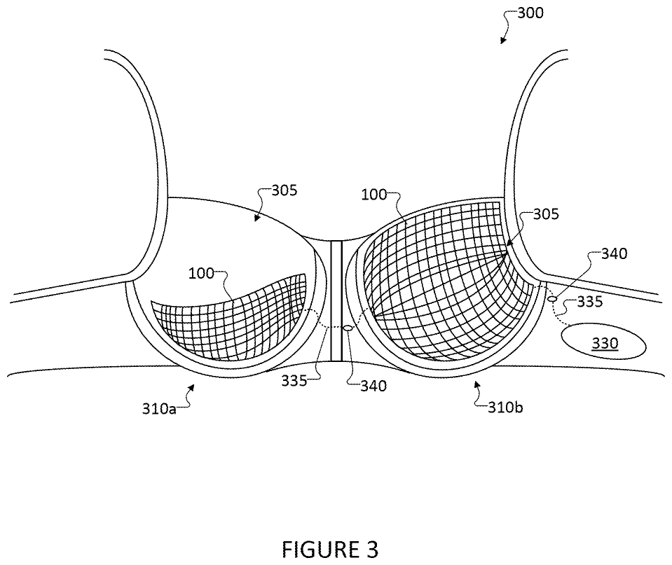

FIG. 3 illustrates a wearable ultrasonic therapy garment according to embodiments of the present disclosure. The embodiment of the wearable ultrasonic therapy garment 300 shown in FIG. 3 is for illustration only. Other embodiments could be used without departing from the scope of the present disclosure.

The wearable ultrasonic therapy garment 300 is configured in the shape of a wearable garment. For example, in the example illustrate in FIG. 3, the wearable ultrasonic therapy garment is configured as a brassiere (bra). The wearable ultrasonic therapy garment 300 is configured to be worn by a user for an extended period of time. The wearable ultrasonic therapy garment 300 is dimensioned to fit particular users. That is, the wearable ultrasonic therapy garment 300 can be dimensioned in different sizes to fit different users. The wearable ultrasonic therapy garment 300 is further dimensioned to be worn under other articles of clothing.

The wearable ultrasonic therapy garment 300 includes a stretchable, breathable, fabric material. The stretchable fabric material can include a knitted design of one or more of: NYLON, POLYESTER, cotton, SPANDEX, LYCRA, or a combination thereof.

The wearable ultrasonic therapy garment 300 is configured to fit a certain part of the body and apply ultrasonic therapy to a designated region corresponding to the certain part of the body. For example, the wearable ultrasonic therapy garment 300 can be a bra configured to apply ultrasonic therapy to one or both breasts subsequent to breast augmentation surgery. Although the example depicted in FIG. 3 illustrates the wearable ultrasonic therapy garment 300 in the form of a bra, other garment forms could be used, such as pants, shorts, shirts, socks, gloves, knee braces, elbow sleeves, and headgear.

In certain embodiments, the wearable ultrasonic therapy garment 300 includes the ultrasound device 100. For example, the wearable ultrasonic therapy garment 300 is disposed in a cup region 305 of the bra. In certain embodiments, the ultrasound device 100 is dimensioned to cover a partial area 310a of the cup region 305 to ensure coverage over an affected or treatment area. In certain embodiments, the ultrasound device 100 can be dimensioned to cover a substantial area 310b of the cup region 305 to ensure full coverage over an affected or treatment area. Regardless of the size of the ultrasound device 100, the ultrasound device 100 includes 100 transducers per 1 cm².

In certain embodiments, the ultrasound device 100 is permanently attached to the wearable ultrasonic therapy garment 300. For example, the ultrasound device 100 can be woven within fibers of the fabric material or sewn onto the fabric material. In certain embodiments, the ultrasound device 100 is affixed to the fabric material by an adhesive such as a silicon adhesive, epoxy, or other glue.

In certain embodiments, the ultrasound device 100 is removably attachable to the wearable ultrasonic therapy garment 300. In certain embodiments, the wearable ultrasonic therapy garment 300 includes one or more pockets configured to hold ultrasound device 100. For example, the wearable ultrasonic therapy garment 300 can have pockets corresponding to the cup regions 305, such as a first pocket to hold a first ultrasound device 100 and a second pocket to hold an ultrasound device 100. In certain embodiments, the ultrasound device 100 includes an adhesive material configured to enable the ultrasound device 100 to be attached to different portions of the wearable ultrasonic therapy garment 300, such as in different portions of the cup regions 305. In certain embodiments, the ultrasound device 100 includes a coupling means such as snaps, buttons, hooks and loops, or adhesives.

In certain embodiments, the wearable ultrasonic therapy garment 300 includes a controller 330. The controller 330 can be the same as, or similar to, controller 230. The controller 330 can be coupled to the ultrasound device 100 via a wireline 335, such as an ACF. In certain embodiments, the wireline 335 includes a number of control lines individually coupled to respective transducers or to respective sets of transducers. In certain embodiments, the controller 330 is removably coupled to the ultrasound device 100. For example, the wireline 335 can include a coupling 340, such as an electrical terminal or data terminal. In certain embodiments, the controller 330 is removably coupled to the wireline 335 and wearable ultrasonic therapy garment 300. For example, the controller 330 can be placed in a pocket in the wearable ultrasonic therapy garment 300 or affixed via an adhesive means, such as hook and loop, button, clips, and the like. The controller 330 can be removable in the event that the ultrasonic therapy sleeve 200 is to be washed.

FIG. 4 illustrates a controller for use with a wearable ultrasonic device according to embodiments of the present disclosure. The embodiment of the controller 400 shown in FIG. 4 is for illustration only. Other embodiments could be used without departing from the scope of the present disclosure.

In certain embodiments, the controller 400 includes a housing 405 and a processor 410. In certain embodiments, the housing 405 comprises a water-tight housing. The processor 410 is configured to control one or more aspects of the ultrasound device 100. For example, the processor 410 can be configured to control a frequency and power output of the ultrasonic energy (namely an ultrasonic signal) emitted by one or more of the piezoelectric transducers of the ultrasound device 100. In certain embodiments, the controller 400 includes a user interface 415 configured to enable user operation of the ultrasound device 100. For example, the user interface 415 can be one or more of: a toggle button, a power button, a mode button, a display, or a touch screen display. In certain embodiments, the user interface 415 includes a speaker configured to emit an auditory signal to the user.

In certain embodiments, the controller 400 includes a connection port 420 configured to removably couple to an external control device. For example, the controller 400 can include a wireline with a connection terminal. In certain embodiments, the controller 400 includes a wireless transceiver 425 configured to wirelessly couple the ultrasound device 100 to an external control device. For example, the controller 400 can establish a communication with the external control device via a near field communication medium, such as BLUETOOTH, ZIGBEE, or the like.

In certain embodiments, the controller 400 includes a power source 430. In certain embodiments, the power source 430 is a battery. In certain embodiments, the power source is a power terminal for connecting to an external power source.

FIG. 5 illustrates an external electronic device 500 for controlling a wearable ultrasonic system according to embodiments of the present disclosure. The embodiment of the external electronic device 500 shown in FIG. 5 is for illustration only. Other embodiments of the external electronic device 500 could be used without departing from the scope of this disclosure.

In various embodiments, the electronic device 500 may take on different forms, and the present disclosure is not limited to any particular form. For example, the electronic device 500 can also be a mobile communication device, such as for example, a user equipment, a mobile device, a mobile station, a subscriber station, a wireless terminal, a smart phone, a tablet, etc.

In the example shown in FIG. 5, the electronic device 500 is capable of coupling to a network environment 505 includes a bus 510, a processor 515, a memory 520, an input/output interface 525, a display 530, and a communication interface 535. In some embodiments of the present disclosure, the electronic device 500 may omit at least one of the above components 510 to 535 or further include other components.

The bus 510 may include, for example, a circuit for connecting the components 515 to 535 of the electronic device 500 and transmitting communication (for example, control messages and/or data) between the components.

The processor 515 may include one or more of a CPU, an AP, a graphic processor unit (GPU) and a communication processor (CP). The processor 515, for example, can carry out operations or data processing relating to control and/or communication of at least one other element of the electronic device 500.

The processor 120 may be referred to as a controller, may include a controller as a part thereof, or may constitute a part of the controller. The controller may control at least one other component of the electronic device 500, for example, the memory 520, the input/output interface 525, the display 530, and the communication interface 535 to perform operations according to a display method according to various embodiments of the present disclosure.

The memory 520 includes a volatile memory and/or a non-volatile memory. The memory 520 may store, for example, commands or data related to at least one of the other components of the electronic device 500. The volatile memory can be used as a buffer for storing one or more layers of the graphic content that can be displayed on the display hardware 530. According to an embodiment of the present disclosure, the memory 520 stores software and/or a program 540. For example, the program 540 includes a kernel 541, middleware 543, an application programming interface (API) 545, and/or application programs (applications, aps or mobile apps) 547. At least some of the kernel 541, the middleware 543, and the API 545 may be referred to as an operating system (OS).

The kernel 541 controls or manages system resources (for example, the bus 510, the processor 515, or the memory 520) used for executing an operation or function implemented by other programs (for example, the middleware 543, the API 545, or the application program 547). Furthermore, the kernel 541 provides an interface through which the middleware 543, the API 545, or the application program 547 may access individual components of the electronic device 500 to control or manage system resources.

The middleware 543 serves as, for example, an intermediary for allowing the API 545 or the application programs 547 to communicate with the kernel 541 to exchange data. Furthermore, with regard to task requests received from the applications 547, the middleware 543 may perform a control for example, scheduling or load balancing for the task requests using, for example, a method of assigning a priority, by which the system resources, for example, the bus 510, the processor 520, or the memory 530 of the electronic device 500 may be preferentially used, to at least one of the applications 547.

The API 545 is, for example, an interface by which the applications 547 control functions provided from the kernel 541 or the middleware 543, and can include, for example, at least one interface or function, such as for example, commands for file control, window control, image processing, or text control.

The input/output interface 525 can serve as an interface capable of delivering a command or data, which is input from a user or another external device, to the component(s) other than the input/output interface 525 within the electronic device 500. Furthermore, the input/output interface 525 outputs the instructions or data received from the other component(s) of the electronic device 101 to the user or another external device.

The display 530 can include a liquid crystal display (LCD), a light-emitting diode (LED) display, an organic LED (OLED) display, a microelectromechanical systems (MEMS) display, and an electronic paper display. For example, the display 530 displays various pieces of content, for example, text, images, videos, icons, symbols, and the like to the user. The display 530 includes a touch screen and receives, for example, a touch, gesture, proximity, or hovering input using an electronic pen or the user's body part.

The communication interface 535 configures communication between, for example, the electronic device 500 and another device such as, a ultrasound device 100, a second external electronic device 550, or a server 555. For example, the communication interface 535 may be connected to a network 505 through wireless or wired communication 560 to communicate with the external device, for example, the second external electronic device 550 or the server 555. In various embodiments, the communication interface 535 can also communicate with an external device, such as the ultrasound device 100, using a wireless communication 560.

In certain embodiments, when coupled to the ultrasound device 100, such as via controller 400, the electronic device 500 is configured to provide electrical energy to the ultrasound device 100. For example, the electronic device 500 can deliver electrical energy to the ultrasound device 100 via the power source 430 of the controller 400.

The wireless communication 560 can include at least one of, for example, Wi-Fi, BLUETOOTH (BT), near field communication (NFC), GPS, and cellular communication (for example, long term evolution (LTE), LTE-advanced (LTE-A), code division multiple access (CDMA), wideband CDMA (WCDMA), universal mobile telecommunication system (UMTS), wireless broadband (WiBro), global system for mobile communications (GSM), and the like). The wired communication can include at least one of, for example, a universal serial bus (USB), a high definition multimedia interface (HDMI), a recommended standard 232 (RS-232), and a plain old telephone service (POTS).

According to an embodiment of the present disclosure, the network 505 includes a telecommunication network, for example, at least one of a computer network (for example, a local area network (LAN) or a wide area network (WAN)), Internet, and a telephone network.

The second external electronic device 550 can be a device that is the same as or different from the electronic device 500. According to an embodiment of the present disclosure, the server 555 can include a group of one or more servers. According to various embodiments of the present disclosure, all or some of the operations performed by the electronic device 500 can be performed by another electronic device or a plurality of electronic devices. For example, all or some of the operations can be performed by one or more of the ultrasound device 100, the second external electronic device 550, or the server 555. According to an embodiment of the present disclosure, when the electronic device 500 should perform some functions or services automatically or by a request, the electronic device 500 makes a request for performing at least some functions related to the functions or services to another device such as the first external electronic device 102, the second external electronic device 104, or the server 106 instead of performing the functions or services by itself. The other electronic device, being the ultrasound device 100, the second external electronic device 550, or the server 555 can carry out the requested function or the additional function, and transfer a result thereof to the electronic device 500. The electronic device 500 can also provide the requested functions or services based on the received result directly or after additional processing of the received result. To achieve this, for example, cloud computing, distributed computing, or client-server computing technology may be used.

According to an embodiment of the present disclosure, the ultrasound device 100 is attachable to and detachable from the electronic device 500. In this case, the ultrasound device 100 may not include a display. For example, when the electronic device 500 is used while being coupled to the ultrasound device 100, the electronic device 500 provides, through the display 530, a screen to the user who wears the ultrasound device 100, to which the electronic device 500 is coupled, to provide information regarding the ultrasound device 100 as well as control one or more functions of the ultrasound device 100.

In certain embodiments, the external electronic device 500 is a remote electronic device capable of connecting to the ultrasound device 100 via network 505. Accordingly, in certain embodiments, controller 400 of ultrasound device 100 is configured to connect to the network 505. For example, in certain embodiments, the electronic device 500 connects to ultrasound device 100 through the internet, such as via an internet protocol (IP) connection. That is, in certain embodiments, the controller 400 is configured to connect to the internet and obtain an IP address. Additionally, the electronic device and or server 555 is able to connect to the internet and obtain an IP address. In certain embodiments, the electronic device 550 is configured to couple to the server 555 and the server 555 is configured to communicate and control the ultrasound device 100 via network 505, such as via the internet.

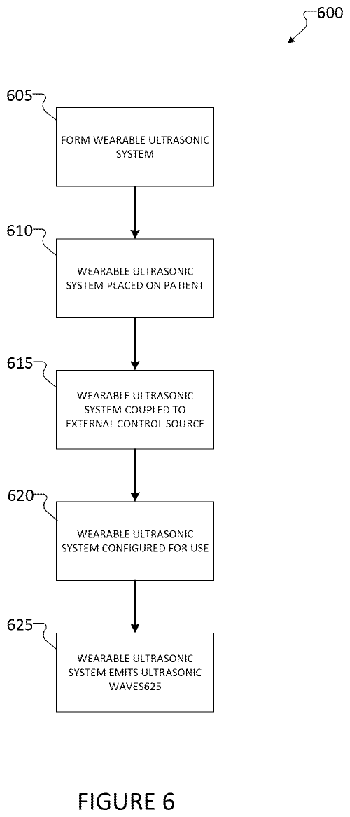

FIG. 6 illustrates a process 600 for providing ultrasonic therapy by a wearable ultrasonic device according to embodiments of the present disclosure. While the flow chart depicts a series of sequential steps, unless explicitly stated, no inference should be drawn from that sequence regarding specific order of performance, performance of steps or portions thereof serially rather than concurrently or in an overlapping manner, or performance of the steps depicted exclusively without the occurrence of intervening or intermediate steps. The process 600 depicted in the example depicted is implemented by a processors and circuitry in, for example, a wearable ultrasound device, a mobile terminal, or a combination thereof

In operation 605, an ultrasound device 100 is placed in a sleeve or garment to form a wearable ultrasonic system. In certain embodiments, the ultrasound wearable device 100 is permanently affixed to the sleeve or garment. In certain embodiments, ultrasound wearable device 100 is removably attached to the sleeve or garment. The wearable ultrasonic system is configured to light weight and portable, enabling free movement of the wearer (patient) as needed.

In operation 610, a wearable ultrasonic system is placed on a patient for treatment. The patient may have recently, i.e., within the last two years, undergone a surgical procedure in which an injury to bone or soft tissue was repaired, or in which a foreign object, such as one or more supporting pins, breast implant, artificial joint, or prosthetic device was placed in the patient. As part of the recovery process, a surgeon, physician, or physical therapist may prescribe therapy to inhibit the occurrence of scar tissue as a result of capsular contracture, arthrofibrosis, or another tissue scarring cause. The wearable ultrasonic system is gently placed on the patient by either sliding into place of affixing into position by the patient, doctor, or therapist.

In operation 615, the wearable ultrasonic system is coupled to a control source for operation. The wearable ultrasonic system may be coupled to an external electronic device, such as a smart phone or tablet computer.

In operation 620, the wearable ultrasonic system is configured for use. In certain embodiments, the patient enters one or more input commands to commence operation of the ultrasound device 100 in accordance with directions and settings specified by the doctor or physical therapist. In certain embodiments, the doctor or therapist remotely enter one or more input commands to commence operation of the ultrasound device 100. For example, a doctor may be able to remotely connect to the smart phone (external electronic device 500) coupled to the wearable ultrasonic system and remotely control operation of the wearable ultrasonic system. In certain embodiments, the wearable ultrasonic system is able to feedback information regarding the wearable ultrasonic system or other data regarding the patient that is collected by the smart phone or wearable ultrasonic system. In certain embodiments, the doctor or therapist saves the setting and operation information for the wearable ultrasonic system on a server. When treatment is to be performed, the smart phone (external electronic device 500) connects to the server, such as via a application on the smart phone, and operates the ultrasound device 100 in accordance with the settings on the server. Thereafter, the doctor or physical therapist can routinely adjust one or more settings for the ultrasonic treatment by varying the settings saved on the server, such as based on information received about the patient or responsiveness of the patient to the therapy.

In operation 625, the wearable ultrasonic system emits ultrasonic waves in accordance with settings input by the patient, doctor, or therapist. While in operation, the patient is able to move around and change positions as desired. The ultrasonic treatment can be applied for the duration specified or desired by the patient, doctor, or therapist. Upon completion, the wearable ultrasonic system can be removed and cleaned for later use.

Therefore, embodiments of the present disclosure provide a portable, light weight ultrasound therapy device. The ultrasound therapy device is configured to be part of a sleeve or garment worn by the patient. The ultrasound therapy device enables a patient to perform ultrasound therapy in the comfort of their own home while ensuring that the therapy is properly applied according to guidelines provided by a medical professional.

Although the present disclosure has been described with various embodiments, various changes and modifications may be suggested to one skilled in the art. It is intended that the present disclosure encompass such changes and modifications as falling within the scope of the claims.

The present disclosure should not be read as implying that any particular element, step, or function is an essential element, step, or function that must be included in the scope of the claims. Moreover, the claims are not intended to invoke 35 U.S.C. § 112(f) unless the exact words “means for” are followed by a participle.

What is claimed is:

1. A system comprising:
a stretchable fabric configured to be worn on a body portion of a patient;
a flexible ultrasound device coupled to the stretchable fabric and configured to:
be non-invasively positioned proximate a skin surface of the patient above a target site below the skin surface; and
emit an ultrasonic energy; and
a control system configured to operate the plurality of transducers to direct the ultrasonic energy into the target site.

2. The system of claim 1, wherein the flexible ultrasound device comprises a plurality of transducers.

3. The system of claim 2, wherein the plurality of transducers is disposed in an N×M grid.

4. The system of claim 2, wherein the flexible ultrasound device is configured to conform to one or more surfaces of the portion of the body.

5. The system of claim 1, wherein the flexible ultrasound device is configured to at least one of:
attach to the stretchable fabric; and
be inserted into the stretchable fabric.

6. The system of claim 1, wherein the control system is detachably coupled to the flexible ultrasound device.

7. The system of claim 1, wherein the control system is configured to at least one of:
communicate via a wireless communication with an external electronic device; or
control cause a first subset of the plurality of transducers to emit the ultrasonic energy while causing a second subset of the plurality of transducers to not emit the ultrasonic energy.

8. A wearable garment comprising:
a fabric housing configured to be worn on a body portion of a patient;
a flexible ultrasound device configured to emit an ultrasonic energy a target site below a skin surface corresponding to the body portion of the patient; and
a control system configured to operate the plurality of transducers to direct the ultrasonic energy into the target site.

9. The wearable garment of claim 8, wherein the flexible ultrasound device comprises a plurality of transducers disposed in an N×M grid.

10. The wearable garment of claim 9, wherein the flexible ultrasound device is configured to conform to one or more surfaces of the portion of the body.

11. The wearable garment of claim 8, wherein the flexible ultrasound device is configured to at least one of:
attach to an inner surface of the fabric housing;
be inserted into a pocket of the fabric housing; or
be woven through one or more fibers of the fabric housing.

12. The wearable garment of claim 8, wherein the control system is detachably coupled to the flexible ultrasound device and removable from the wearable garment.

13. The wearable garment of claim 8, wherein the control system is configured to at least one of:
communicate via a wireless communication with an external electronic device; or
control cause a first subset of the plurality of transducers to emit the ultrasonic energy while causing a second subset of the plurality of transducers to not emit the ultrasonic energy.

14. The wearable garment of claim 8, where the wearable garment comprises one of:
a knee brace;
an elbow brace;
an ankle brace;
a foot brace;
a hand brace;
a shoulder brace;
a back posture brace;
a hip wrap;
a hip brace; or
a brassiere.

15. A method comprising:
providing a flexible ultrasound device to be positioned on a surface of a body of a patient by a wearable garment, the flexible ultrasound device comprising a plurality of transducers; and
controlling an operation of the plurality of transducers to direct the ultrasonic energy into a target site beneath a skin of the patient.

16. The method of claim 15, wherein the flexible ultrasound device comprises a plurality of transducers disposed in an N×M grid.

17. The method of claim 16, further comprising conforming, via the wearable garment, the plurality of transducers to one or more surfaces of the portion of the body.

18. The method of claim 15, transmitting, from an external electronic device, a wireless signal to a control system of the flexible ultrasound device to control the operation of the plurality of transducers.

19. The method of claim 18, wherein the external electronic device is one of:
a portable electronic device; or
a remote electronic device.

20. The method of claim 18, wherein the external electronic device is configured to specify one or more of:
a duration of operation;
a repetition of operation; or
activation of a first subset of the plurality of transducers to emit the ultrasonic energy while causing a second subset of the plurality of transducers to not emit the ultrasonic energy.
